(19) United States
(12) Patent Application Publication
(10) Pub. No.: US 2026/0216021 A1
(43) Pub. Date: Jul. 30, 2026

(54) ORAL CARE COMPOSITIONS COMPRISING 3-PHENYL-1-PROPANOL AND AN ALKANEDIOL AND 4-ISOPROPYL-3-METHYLPHENOL

(71) Applicant: SYMRISE AG, Holzminden (DE)

(72) Inventors: Steffen NORDZIEKE, Göttingen (DE); Laura MEUNIER, Einbeck (DE); Florian GENRICH, Dassel (DE)

(21) Appl. No.: 19/149,132

(22) PCT Filed: Feb. 10, 2023
(86) PCT No.: PCT/EP2023/053375

§ 371 Date: Jul. 18, 2025

(51) Int. Cl. A61K 8/34; A61K 8/35; A61Q 11/00

(52) U.S. Cl. CPC A61K 8/347; A61K 8/35; A61Q 11/00; A61K 2800/92

(57) ABSTRACT

The present invention relates to oral care compositions comprising 3-phenyl-1-propanol, an alkanediol, preferably decylene glycol and 4-isopropyl-3-methylphenol to treat bacterial and fungal infections in the mouth and to reduce oral malodour.

### TECHNICAL FIELD

The present invention relates to oral care compositions comprising 3-phenyl-1-propanol, an alkanediol, preferably decylene glycol, and 4-isopropyl-3-methylphenol to treat bacterial and fungal infections in the mouth and to reduce oral malodour.

### BACKGROUND ART

Oral malodour is primarily caused by enzymatic degradation of proteins and amino acids. Typically formed malodorous components comprise volatile sulphur compounds, such as hydrogen sulphide, methyl mercaptan and dimethyl sulphide; indole and skatole. Often, higher abundances of anaerobic gram-negative bacteria, such as Porphyromonas and Fusobacterium, are correlated to stronger malodour.

Apart from oral malodour, bacteria and fungi can have a further negative impact on the oral hygiene as they can cause tooth infections, dental caries, periodontal infections, halitosis, gingivitis, candidiasis and other infections, in particular those induced by anaerobic gram-negative bacterial strains.

Oral malodour is therefore not desirable for both cosmetic and medical reasons, since an unpleasant odour can typically also indicate infections in the mouth; thus, efforts have been made to improve the oral hygiene, for example by using mouthwashes or mouth rinses.

Oral care compositions used for the reduction of oral malodour typically contain antimicrobial compounds, such as 4-isopropyl-3-methylphenol, as disclosed in WO 2008/126057.

According to WO 2013/08762, 4-isopropyl-3-methylphenol may also serve as anti-inflammatory agent in compositions which are designed for oral care applications. Mouthwash formulations comprising 4-isopropyl-3-methylphenol are furthermore disclosed in WO 2021/074164.

An antibacterial system comprising 4-isopropyl-3-methylphenol, zinc ions and an anionic surfactant is disclosed in WO 2010/112577. The invention can also be used in mouthwash compositions and is effective against bacterial strains present in the mouth, as shown by in vitro studies.

### Technical Problem

Oral malodour is a problem in general and is related to mouth diseases, which are even more problematic.

The present invention has been made in view of the problem mentioned above and an object of the present invention is to determine compositions or applications that can reduce mouth malodour and prevent oral diseases in general.

These issues are often related to bacteria, and thus a further secondary problem of the invention is to establish compositions that reduce the amount of bacteria in the oral cavity.

### Solution to Problem and Advantages of the Invention

The present invention is an oral care composition comprising 3-phenyl-1-propanol, an alkanediol or alkanediol mixtures, which is preferably decylene glycol, and 4-isopropyl-3-methylphenol. Surprisingly, it was found that the aforementioned composition strongly reduces the amount of bacteria and fungi in the mouth and thus is capable of treating oral infections. However, the composition is also active against fungi. In addition, the three substances in combination are more effective in reducing oral malodour.

The effects of the invention have been examined by in vivo experiments. In particular, the improved antibacterial properties of the oral care composition compared to the state of the art have been demonstrated. In addition, the oral care composition has antifungal properties.

Preferably, the above alkanediol or alkanediol mixture that forms part of the composition is/are selected from the group of linear 1,2-diols, 2,3-diols or 4,5-diols. Further preferable is that said alkanediols have a chain length of five to twenty carbon atoms. Preferably, the alkanediol is selected from the group consisting of 1,2-pentanediol, 1,2-hexanediol, 1,2-heptanediol, 1,2-octanediol, 1,2-nonanediol and 1,2-decanediol, also preferably the alkanediol is 2,3-hexanediol and/or 2,3-heptanediol and/or 2,3-octanediol. Further preferably the alkanediol is a combination of 1,2-hexanediol and 2,3-hexanediol, or 1,2-heptanediol and 2,3-heptanediol, or 1,2-octanediol and 2,3-octanediol. More preferably, the alkanediol comprises 1,2-octanediol or 1,2-decanediol, also preferably or 1,2-hexanediol, 1,2-octanediol and/or 1,2-decanediol. Most preferred is that the alkanediol is 1,2-decanediol.

In a further more preferred variant, the above alkanediol is selected from the group consisting of alkanediols having a lower number of carbon atoms of 5 to 8, i.e. pentanediol, hexanediol, heptanediol, octanediol, and any mixture thereof, or is selected from the group consisting of alkanediols having a number of carbon atoms of 6 to 8, i.e. hexanediol, heptanediol, octanediol, and any mixture thereof, or is selected from the group consisting of alkanediols having a number of carbon atoms of 7 and 8, i.e. heptanediol, octanediol, and any mixture thereof.

In a still more preferred variant, the alkanediol is selected from the group consisting of alkanediols having a higher number of carbon atoms of 9 to 12, i.e. nonanediol, decanediol, undecanediol, dodecanediol, and any mixture thereof, or is selected from the group consisting of alkanediols having a number of carbon atoms of 9 to 11, i.e. nonanediol, decanediol, undecanediol, and any mixture thereof, or is selected from the group consisting of alkanediols having a number of carbon atoms of 9 and 10, i.e. nonanediol, decanediol, and any mixture thereof.

In a still more preferred variant, the alkanediol is either pentanediol, hexanediol, heptanediol or octanediol, or any mixture thereof. In a still more preferred variant, the alkanediol is either nonanediol, decanediol, undecanediol or dodecanediol, or any mixture thereof. In a most preferred variant, the alkanediol is decanediol.

The term “alkanediol” within the context of the present invention also includes its constitutional isomers or position isomers. Constitutional isomers are compounds that have the same molecular formula and different connectivity. Position isomers, a particular form of constitutional isomerism, are structural isomers that can be viewed as differing only on the position of a functional group on a parent structure, which in this case, in particular, is the position of the two alcohol functions.

Depending on the number of the carbon atoms of the carbon chain of the alkanediol, there are various position isomers of the alkanediol: (x,x+1) constitutional isomers; (x,x+2) constitutional isomers; (x,x+3) constitutional isomers; etc. and alpha, omega constitutional isomers when the alcohol functions are at the terminal ends of the carbon chain, wherein x stands for the number of the carbon atom in the alkanediol chain, to which the OH groups of the alkanediol are chemically bonded. For example: if x is 1, the two OH groups of the alkanediol are chemically bonded to the C1 and C2 carbon atoms in the alkanediol chain; if x is 2, the two OH groups of the alkanediol are chemically bonded to the C2 and C3 carbon atoms in the alkanediol chain, etc.

In the (x,x+1) constitutional isomers, the two OH functional groups are vicinal attached to two different adjacent carbon atoms in the chain. In the (x,x+2) constitutional isomers, the two OH functional groups are attached to two different carbon atoms in the chain where the two carbon atoms are separated by one C atom. In the (x,x+3) constitutional isomers, the two OH functional groups are attached to two different carbon atoms in the chain where the two carbon atoms are separated by two C atoms. In the alpha, omega constitutional isomers, the two functional groups are attached to the first C atom and to the terminal C atom.

In a preferred variant of the oral care composition, the alkanediol having a carbon chain of 5 to 12 carbon atoms, is preferably a vicinal (x,x+1) diol, selected from the group consisting of a 1,2-diol, 2,3-diol, 3,4-diol, 4,5-diol, further (x,x+1) diols, and mixtures thereof, preferably an alpha, beta-1,2-constitutional isomer.

Thus, according to a preferred variant of the oral care composition, the alkanediol is an (x,x+1) constitutional isomer, wherein x stands for the number of the carbon atom in the alkanediol chain, to which the OH groups of the alkanediol are chemically bonded, in particular a 1,2-alkanediol, a 2,3-alkanediol, a 3,4-alkanediol, or mixtures thereof, preferably a 1,2-alkanediol or a 2,3-alkanediol.

In a further preferred variant of the oral care composition according to the present invention, the alkanediol having a carbon chain of 5 to 12 carbon atoms, is preferably a non-vicinal (x,x+2) diol, selected from the group consisting of a 1,3-diol, 2,4-diol, 3,5-diol, further (x,x+2) diols, and mixtures thereof, preferably an alpha, gamma-1,3-constitutional isomer.

In a preferred variant of the oral care composition, the alkanediol having a carbon chain of 5 to 12 carbon atoms is a non-vicinal (x,x+2) diol, selected from the group consisting of a 1,3-diol, 2,4-diol, 3,5-diol, 4,6-diol, further (x,x+2) diols, and mixtures thereof, preferably an alpha, gamma-1,3-constitutional isomer.

In a further preferred variant of the oral care composition the alkanediol having a carbon chain of 5 to 12 carbon atoms, is preferably a non-vicinal (x,x+3) diol, selected from the group consisting of a 1,4-diol, 2,5-diol, further (x,x+3) diols, and mixtures thereof, preferably an alpha, delta-1,4-constitutional isomer.

In a preferred variant of the oral care composition, the alkanediol is preferably an alpha, omega alkanediol, more preferably, 1,7-heptanediol or 1,8-octanediol.

According to the present invention, vicinal (x,x+1) diols are most preferred, such as alpha, beta or beta, gamma or gamma, delta etc.

In a still more preferred variant of the oral care composition, the alkanediol is a 1,2-alkanediol, a 2,3-alkanediol, a 3,4-alkanediol, or mixtures thereof, more preferred 2,3-alkanediol.

In a preferred variant of the oral care composition, the alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
   and any mixture thereof.

According to a preferred variant, in the oral care composition, the alkanediol is selected from the group consisting of 1,2-pentanediol, 2,3-pentanediol, 1,2-hexanediol, 2,3-hexanediol, 1,2-heptanediol, 2,3-heptanediol, 1,2-octanediol, 2,3-octanediol, 1,2-nonanediol, 2,3-nonanediol, 1,2-decanediol, 2,3-decanediol, 1,2-undecanediol, 2,3-undecanediol, 1,2-dodecanediol, 2,3-dodecanediol, and any mixture thereof.

Of the aforesaid linear alkanediols, the following (x,x+1) constitutional isomers are preferred: 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, 2,3-dodecanediol, or mixtures thereof. Said alkanediols are liquid at a purity of 90 to 99%.

Of the aforesaid liquid alkanediols 1,2-pentanediol, 2,3-pentanediol, 1,2-hexanediol, 2,3-hexanediol, 1,2-heptanediol, 2,3-heptanediol, 2,3-octanediol, 2,3-nonanediol, or mixtures of said liquid alkanediols are particularly preferred.

In a further preferred variant of the oral care composition, the alkanediol is selected from the group consisting of:
1,2-pentanediol, 2,3-pentanediol,
1,2-hexanediol, 2,3-hexanediol,
1,2-heptanediol, 2,3-heptanediol,
1,2-octanediol, 2,3-octanediol,
1,2-nonanediol, 2,3-nonanediol,
1,2-decanediol, 2,3-decanediol,
1,2-undecanediol, 2,3-undecanediol,
1,2-dodecanediol, 2,3-dodecanediol,
   and any mixture thereof.

More preferred, the alkanediol is selected from the group consisting of 1,2 and 2,3 mixtures:
1,2-pentanediol and 2,3-pentanediol,
1,2-hexanediol and 2,3-hexanediol,
1,2-heptanediol and 2,3-heptanediol,
1,2-octanediol and 2,3-octanediol,
1,2-nonanediol and 2,3-nonanediol,
1,2-decanediol and 2,3-decanediol,
1,2-undecanediol and 2,3-undecanediol,
1,2-dodecanediol and 2,3-dodecanediol,
   and any mixture thereof.

Further preferred in the oral care composition the alkanediol mixture is a combination of 1,2-heptanediol and 2,3-heptanediol, or 1,2-decanediol and 2,3-decanediol.

Further preferred in the oral care composition, the alkanediol is selected from the group consisting of 1,2-pentanediol, 2,3-pentanediol, and mixtures thereof, or is selected from the group consisting of 1,2-hexanediol, 2,3-hexanediol, and mixtures thereof. However, the linear alkanediol or the first linear alkanediol can also preferably be selected from the group consisting of 1,2-octanediol, 2,3-octanediol, and mixtures thereof.

The oral care composition preferably comprises 3-phenyl-1-propanol, phenoxyethanol or methylbenzyl alcohol, more preferably the composition comprises 3-phenyl-1-propanol.

Alternative antimicrobial agents used in the sense of the invention preferably comprise isomers of isopropylmethylphenol, i.e. 4-isopropyl-3-methlyphenol (o-cymen-5-ol), 2-isopropyl-5-methylphenol (thymol), 3-isopropyl-5-methylphenol (m-cymen-5-ol) and 5-isopropyl-2-methylphenol (carvacrol).

In one aspect of the invention, the oral care composition comprises 4-isopropyl-3-methylphenol, preferably along with other compounds, selected from the groups of flavourings, sweeteners and/or further additives.

In a preferred further development, the oral care composition according to the invention is used for the treatment and/or prevention and/or reduction of oral malodour, preferably in cosmetic mouthwash applications.

In the sense of the present invention, cosmetic mouthwash applications are to be understood as to provide a temporarily control of bad breath or unpleasant mouth feel. After the use of a cosmetic mouthwash, a fresh and pleasant taste is also obtained. However, a cosmetic mouthwash typically does not provide a long-term control of bacteria associated with oral malodour, i.e. bacteria are not killed to a sufficiently large extent.

In a preferred aspect of the present invention, the oral care composition comprises 4-isopropyl-3-methylphenol in an amount of from 0.01 to 0.2 wt.-%, preferably 0.03 to 0.08 wt.-% of the overall composition. Lower concentrations of 4-isopropyl-3-methylphenol were found to be less or insufficiently effective, whereas higher amounts than those given in the aforementioned ranges in some instances could result in problems regarding, for example, solubility, taste and smell of the oral care composition. In addition, a higher concentration conflicts with the limit of the compound allowed for the use in cosmetics.

In another aspect, the oral care composition according to the invention comprises 3-phenyl-1-propanol, an alkanediol, preferably decylene glycol, and 4-isopropyl-3-methylphenol in sum in an amount of from 0.01 to 0.5 wt.-% of the overall composition. The three components work together to afford a particularly pronounced anti-odour advantage. They are also together more effective in reducing bacterial count as shown in FIGS. 5 and 6.

In a further development, the oral care composition of the present invention additionally comprises an antioxidant, preferably p-hydroxyacetophenone. Advantageously, p-hydroxyacetophenone is present in an amount of from 0.05 to 1.0 wt.-%. The antioxidant has a boosting effect on the efficacy of the oral care composition as shown in FIG. 9.

Furthermore, alkanediols provide surprising synergies in combination with the aforementioned compositions, in particular the mixture of comprising 3-phenyl-1-propanol, an alkanediol, preferably decylene glycol, and 4-isopropyl-3-methylphenol. In combination alkanediols can boost the effect in cosmetic compositions to prevent and/or reduce malodours.

Preferably the above mixture comprises an additional secondary linear alkanediol, which is more preferably a linear 1,2-alkanediol having five to twelve carbon atoms are used for this purpose, preferably 1,2-pentanediol, 1,2-hexanediol, 1,2-heptanediol, 1,2-octanediol, 1,2-nonanediol, 1,2-decanediol, 1,2-undecanediol or 1,2-dodecanediol. Apart from the 1,2 C5 to C12 diols, also the 2,3-alkanediols are preferred, in particular 2,3-hexanediol or 2,3-heptanediol are preferably used as the secondary additional alkanediol.

Furthermore, the additional secondary alkanediol can itself be a mixture of alkanediols thus forming a ternary or quaternary alkanediol mixture in the overall oral care composition.

Finally, the additional secondary alkanediol is preferably any of the alkanediols or alkanediol mixtures as mentioned above for the primary alkanediol or alkanediol mixture.

In a further preferred aspect of the present invention, the oral care composition comprises the additional alkanediol or alkanediol mixture in sum in an amount of from 0.01 to 5.0 wt.-%.

Hydrophilic additives also provide surprising synergies when they are added to the aforementioned compositions, as they improve the solubility of the components in aqueous solvents. Additives for this purpose are preferably selected from the group consisting of alcohols, diols, polyols, phenols or esters with good solubilizing properties, preferably selected from the group consisting of ethanol, 1-propanol, isopropanol, phenoxyethanol, benzyl alcohol, ethylene glycol, propylene glycol, 1,3-propanediol, butylene glycol, pentylene glycol, 1,2-hexanediol, hexylene glycol, dipropylene glycol, ethoxydiglycol, propylene carbonate, glycerine carbonate, butylene carbonate, triethyl citrate, ethyl lactate, butyl lactate, ethyl acetate, diethylmalonate, diacetin, dimethyl isosorbide, diethylene glycol, PPG-3 methyl ether or any of their mixtures.

In a further aspect of the present invention, the oral care composition preferably comprises hydrophilic additives, selected from propylene glycol, glycerol, 1,3-propanediol, butylene glycol, dipropylene glycol and ethanol. More preferably, the composition comprises propylene glycol and/or glycerol in sum in an amount of from 0.5 to 20 wt.-%.

In oral care compositions, flavourings are used for several purposes, such as masking an unpleasant taste of active ingredients or providing a fresh breath. As shown in FIG. 7, flavouring mixtures, such as Optamint®, can also be active against microbial strains. Flavourings typically used in oral care compositions comprise compounds or compound mixtures exhibiting, for example, a taste of menthol, mint, citrus or rose. In some cases, the flavour of an oral care composition is also defined as a fragrance. Preferably, the flavouring comprises one or more compounds selected from L-menthol; trans-menthone; (E)-anethole; 5-methyl-2-(1-methylethyl)cyclohexyl lactate; menthol; L-menthan-3-one; cineole; isomenthone; menthyl acetate; 2-isopropyl-N,2,3-trimethylbutyramide; 2-hydroxypropyl (5-methyl-2-propan-2-ylcyclohexyl) carbonate; 2-hydroxyethyl (5-methyl-2-propan-2-ylcyclohexyl) carbonate; (+)-neomenthol; 4,5,6,7-tetrahydro-3,6-dimethylbenzofuran; (S)-p-mentha-1,8-diene; (−)-menthyl lactate; thymol; camphene; pin-2 (10)-ene; (R)-p-mentha-1,8-diene; pin-2 (3)-ene and/or p-mentha-1,3-diene.

In a further development, the oral care composition according to the invention preferably comprises at least one flavouring compound or fragrance, wherein the flavouring is preferably present in an amount of from 0.1 to 1.0 wt.-%.

As shown in FIG. 8, a synergistic effect regarding the microbial activity is achieved, if the flavouring mixture Optamint® is combined with the oral care composition according to the invention.

In oral care compositions, sweeteners are used to mask an unpleasant taste of the composition. However, in the sense of an oral care application, sweeteners should only be added in small amounts. Compounds suitable for the use as sweetener in oral care compositions comprise, for example, sorbitol or saccharine sodium.

In a further aspect of the present invention, the oral care composition preferably comprises a sweetener, preferably the sodium salt of saccharine in an amount of from 0.01 to 0.5 wt.-%.

In another aspect of the present invention, the oral care composition is used as a cosmetic or a medicament, i.e. a therapeutic mouthwash.

In the sense of the present invention, a therapeutic mouthwash is to be understood as a mouthwash containing active chemical or biological ingredients. Thus, a therapeutic mouthwash is intended to help control or reduce disease conditions such as bad breath (halitosis), gingivitis and/or tooth decay by effectively killing bacteria associated with the aforementioned conditions.

In a preferred aspect, the oral care composition according to the present invention is preferably used to treat and/or prevent oral bacterial and fungal infections, preferably tooth infections and inflammations, tooth decay, dental caries, periodontal infections and inflammations, halitosis, gingivitis, candidiasis and/or infections with gram-negative bacterial strains, preferably F. nucleatum and P. gingivalis.

The abundance of bacteria and fungi in the mouth is generally determined by the collection of oral saliva. Preferably, the collected oral saliva sample is placed in a sample collection tube, and the biological sample is placed in a sterile EP tube. An appropriate amount of lysis buffer is added to the EP tube and mixed, then a bacterial DNA extraction kit is used to extract bacterial genomic DNA from the samples. The quantitative evaluation is preferably completed using Q-PCR.

In a preferred aspect of the present invention, the oral care composition preferably reduces the abundance of the gram-negative bacterial strains F. nucleatum and P. gingivalis in the mouth by at least 20% after 4 weeks, preferably by at least 40% after 4 weeks and more preferably by at least 80% after 4 weeks as examined by the collection of oral saliva. Most preferably, the abundance of the bacterial strains is reduced by 95 or more %.

Oral malodour is primarily caused by enzymatic degradation of proteins and amino acids. Typically formed malodorous components comprise volatile sulphur compounds, such as hydrogen sulphide, methyl mercaptan and dimethyl sulphide. The aforementioned compounds can preferably be detected in the breath using a portable sulphide detector, commercially known and available as Halimeter©.

In another aspect the oral care composition according to the present invention preferably reduces the oral abundance of volatile organosulphur compounds, comprising methyl mercaptan and dimethyl sulphide by at least 30% after 4 weeks, preferably by at least 40% after 4 weeks and more preferably by at least 65% after 4 weeks as examined by a sulphide detector (Halimeter©). Most preferably, the abundance of volatile organosulphur compounds is reduced by 95% or more than 95%.

In a further aspect, the oral care composition according to the invention is preferably used as a mouthwash, cosmetic mouthwash, therapeutic mouthwash, toothpaste component, tooth gel component, subgingival gel component or mouth odour reducing agent comprising the use in mouth sprays, chewable tablets and/or chewing gums.

### BRIEF DESCRIPTION OF DRAWINGS

FIG. 1 is a diagram showing average concentrations of methyl mercaptan in the breath of test persons using either a mouthwash composition according to the invention or a placebo mouthwash composition. The diagram shows the concentrations of methyl mercaptan after 0 days, 7 days and 28 days and includes the corresponding standard deviations.

FIG. 2 is a diagram showing average concentrations of dimethyl sulphide in the breath of test persons using either a mouthwash composition according to the invention or a placebo mouthwash composition. The diagram shows the concentrations of dimethyl sulphide after 0 days, 7 days and 28 days and includes the corresponding standard deviations.

FIG. 3 is a diagram showing average concentrations of the bacterial strain P. gingivalis in the oral saliva of test persons using either a mouthwash composition according to the invention or a placebo mouthwash composition. The diagram shows the concentrations of P. gingivalis after 0 days and 28 days and includes the corresponding standard deviations.

FIG. 4 is a diagram showing average concentrations of the bacterial strain F. nucleatum in the oral saliva of test persons using either a mouthwash composition according to the invention or a placebo mouthwash composition. The diagram shows the concentrations of F. nucleatum after 0 days and 28 days and includes the corresponding standard deviations.

FIG. 5 is a diagram showing the antimicrobial efficacy of a composition containing only the antimicrobial agent 4-isopropyl-3-methylphenol.

FIG. 6 is a diagram showing the antimicrobial efficacy of a composition containing the antimicrobial agent 4-isopropyl-3-methylphenol together with the 1,2-alkanediol decylene glycol and 3-phenyl-1-isopropanol.

FIG. 7 is a diagram showing the antimicrobial efficacy of a composition containing the flavouring mixture Optamint®.

FIG. 8 is a diagram showing the antimicrobial efficacy of a composition containing the flavouring mixture Optamint®, 4-isopropyl-3-methylphenol, 3-phenyl-1-propanol and decylene glycol.

FIG. 9 is a diagram showing the antimicrobial efficacy of a composition containing the flavouring mixture Optamint®, 4-isopropyl-3-methylphenol, 3-phenyl-1-propanol, decylene glycol and 4-hydroxyacetophenone.

### EXPERIMENTAL PART

The influence of the oral care composition according to the invention on the oral malodour was determined by a sulphide detector (Halimeter®) and the evaluation of relevant bacterial strains from oral saliva.

The oral care composition comprising 4-isopropyl-3-methylphenol, 3-phenyl-1-propanol and decylene glycol (Sample 2) was prepared according to Tab. 1. The corresponding placebo composition (Sample 1) is also described in Tab. 1.

TABLE 1
Oral care composition comprising 4-isopropyl-3-methylphenol,
3-phenyl-1-propanol and decylene glycol (Sample 2) and
placebo (Sample 1).

| | wt.-% Material | |
|---|---|---|
| INCI | Sample 1 | Sample 2 |
| WATER | 92.05 | 90.55 |
| PROPYLENE GLYCOL | 2.00 | 2.00 |
| HYDROXYACETOPHENONE | 0.30 | 0.30 |
| 1,2-HEXANEDIOL, | 0.50 | 0.50 |
| CAPRYLYL GLYCOL | | |
| GLYCERIN | 4.00 | 4.00 |
| PEG-40 HYDROGENATED CASTOR OIL | 0.80 | 2.00 |
| FLAVOUR | 0.30 | 0.30 |
| PHENYLPROPANOL, | — | 0.30 |
| o-CYMEN-5-OL, DECYLENE GLYCOL | | |
| SODIUM SACCHARIN | 0.05 | 0.05 |

The oral care composition according to the invention (Sample 2) and the placebo composition (Sample 1) were administered to 35 healthy test persons in the form of a mouthwash twice a day (morning and evening) for 28 days. The breath of the subjects was evaluated by a sulphide detector on the first day of the test and after 7 and 28 days of use. Oral saliva was collected on the first day of the test and after 28 days of use.

The results of the breath analysis of the test persons using a portable sulphide detector (Halimeter©) are summarised in Tab. 2. The table shows the average oral ppb-concentrations of the volatile organosulphur compounds hydrogen sulphide (H₂S), methyl mercaptan (CH₃SH) and dimethyl sulphide (CH₃SCH₃) together with the corresponding standard deviations.

TABLE 2
Results of the analysis of the breath of test persons for volatile
organosulphur compounds over time using a portable sulphide
detector (Halimeter ®)

| Compound | | Averages and standard deviations [ppb] | |
|---|---|---|---|
| analysed | Time | Sample 1 | Sample 2 |
| Hydrogen sulphide | 0 days | 438.62 ± 428.41 | 508.18 ± 308.60 |
| (H₂S) | 7 days | 445.62 ± 451.62 | 371.65 ± 400.51 |
| | 28 days | 648.69 ± 658.14 | 474.29 ± 296.73 |
| Methyl mercaptan | 0 days | 155.25 ± 132.70 | 233.76 ± 316.20 |
| (CH₃SH) | 7 days | 173.88 ± 186.44 | 114.94 ± 135.63 |
| | 28 days | 212.50 ± 232.66 | 114.65 ± 86.79 |
| Dimethyl sulphide | 0 days | 105.19 ± 108.53 | 113.18 ± 68.15 |
| (CH₃SCH₃) | 7 days | 62.75 ± 61.88 | 24.82 ± 19.70 |
| | 28 days | 197.81 ± 257.32 | 53.82 ± 49.06 |

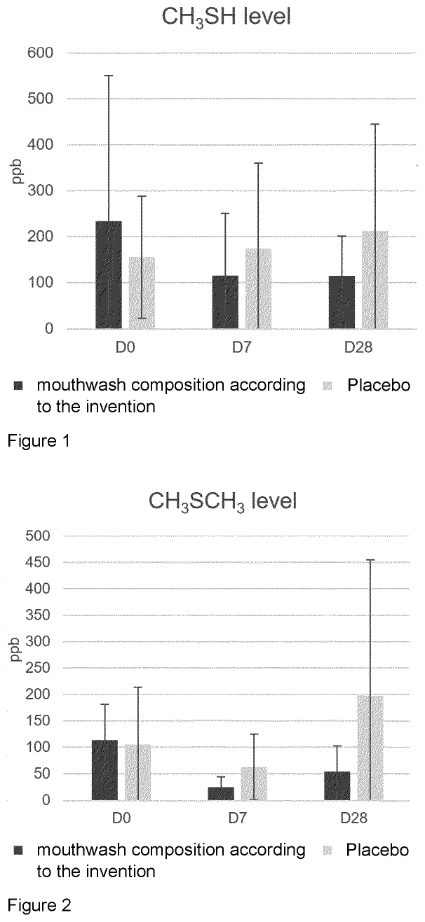

As can be seen from the data in Tab. 2, Sample 2 reduces the amount of volatile organosulphur compounds present in the mouth. For hydrogen sulphide, the effect is only weakly pronounced, however, for methyl mercaptan (see also FIG. 1) and dimethyl sulphide (see also FIG. 2) a clear decrease in the concentrations of the volatile sulphur compounds is observed. In contrast, no decrease is observed when Sample 1 is used as mouthwash.

The procedure for the evaluation of the oral saliva was carried out as follows. The collected oral saliva sample is placed in a sample collection tube, and the biological sample is placed in a sterile EP tube, and then an appropriate amount of lysis buffer is added to the EP tube and mixed evenly. The bacterial DNA extraction kit extracts bacterial genomic DNA from clinical samples. Q-PCR was used to complete the quantitative evaluation of Streptococcus mutans, P. gingivalis and F. nucleatum.

The results of the oral saliva analysis of the test persons are summarised in Tab. 3. The table shows the average oral ppm-concentrations of the bacterial strains Streptococcus mutans, P. gingivalis and F. nucleatum together with the corresponding standard deviations.

TABLE 3
Results of the oral saliva analysis of test persons strain

| Bacterial strain | | Averages and standard deviations [ppm] | |
|---|---|---|---|
| analysed | Time | Sample 1 | Sample 2 |
| Streptococcus | 0 days | 527.94 ± 977.86 | 857.69 ± 1188.56 |
| mutans | 28 days | 960.67 ± 1095.95 | 996.47 ± 1393.34 |
| P. gingivalis | 0 days | 657.94 ± 504.43 | 656.81 ± 735.43 |
| | 28 days | 726.00 ± 666.46 | 484.44 ± 624.59 |
| F. nucleatum | 0 days | 355.25 ± 232.57 | 454.31 ± 362.35 |
| | 28 days | 246.25 ± 228.06 | 253.25 ± 170.62 |

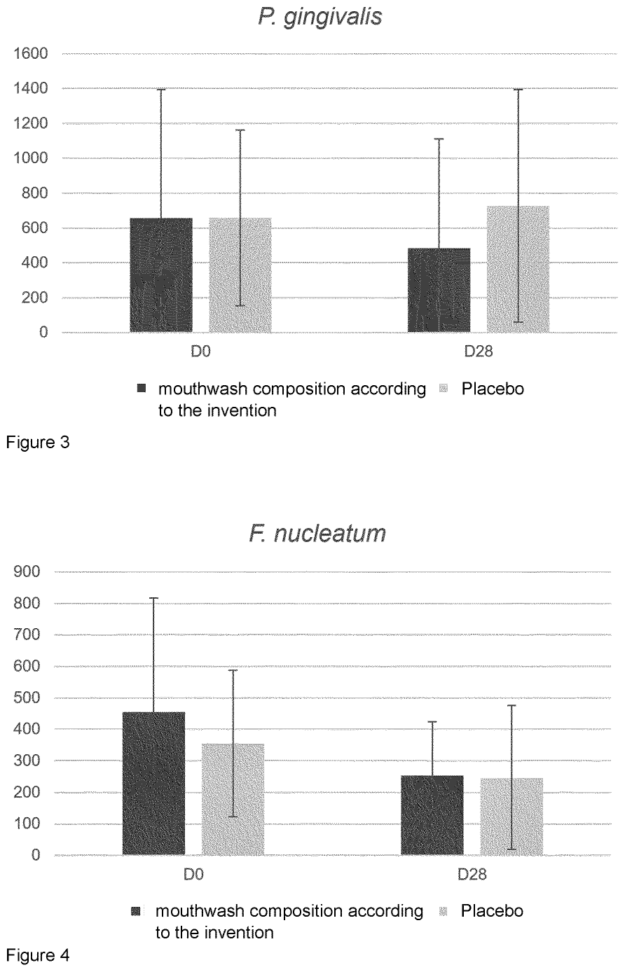

As can be seen from the data in Tab. 3, Sample 2 reduces the amount of bacteria in the mouth, in particular P. gingivalis and F. nucleatum (see also FIGS. 3 and 4). However, Sample 2 is not as effective against Streptococcus mutans, although a relatively less increase can be seen compared to the placebo (Sample 1). As expected, the placebo composition does not reduce the amount of bacteria in the mouth.

### Comparative Example 1: Synergistic Effect Between 4-isopropyl-3-methylphenol, 3-phenyl-1-propanol and Decylene Glycol

In another experiment, the antimicrobial efficacy of the composition according to the invention was compared to the efficacy of 4-isopropyl-3-methylphenol alone. For this purpose, the sample compositions 3 and 4 were prepared according to Tab. 4.

The amount of 0.20 wt.-% of the mixture 3-phenyl-1-propanol, 4-isopropyl-3-methylphenol and decylene glycol in Sample 4 corresponds to an amount of 0.04 wt.-% of pure 4-isopropy-3-methylphenol. Thus, Sample 3 contains significantly more antimicrobial agent than Sample 4.

TABLE 4
Sample composition comprising 4-isopropyl-3-methylphenol,
3-phenyl-1-propanol and decylene glycol (Sample 4) and
sample composition comprising 4-isopropyl-3-
methylphenol (IPMP) alone (Sample 3).

| | wt.-% Material | |
|---|---|---|
| EU INCI | Sample 3 | Sample 4 |
| Aqua | 82.10 | 82.00 |
| Carbomer | 0.20 | 0.20 |
| Stearyl heptanoate, stearyl | 4.00 | 4.00 |
| caprylate | | |
| Ethylhexyl isononanoate | 5.00 | 5.00 |
| Stearyl alcohol | 2.00 | 2.00 |
| Glyceryl stearyl citrate | 3.00 | 3.00 |
| Sodium hydroxide, aqua | 0.30 | 0.30 |
| o-Cymen-5-ol | 0.10 | — |
| Phenylpropanol, | — | 0.20 |
| o-cymen-5-ol, decylene glycol | | |
| Hydroxyacetophenone | 0.30 | 0.30 |
| Dipropylene glycol | 3.00 | 3.00 |

Sample 3 was prepared as follows. Stearyl heptanoate, stearyl caprylate, ethylhexyl isononanoate and stearyl alcohol were heated to approx. 80° C. to form Phase A. In parallel, glyceryl stearate citrate and water were also added to approx. 80° C. to form Phase B. Carbomer was added to Phase A and homogenized, then Phase B was added to Phase A and emulsified using an Ultra Turrax stirrer. The emulsification step was carried out over a period of 2 minutes with a rotation speed of 4000 min-1. The obtained emulsion was allowed to cool down using a vane stirrer. In a separate vessel, 4-isopropyl-3-methylphenol, 4-hydroxyacetophenone and dipropylene glycol were premixed and heated up to 80° C. to form Phase D. The premixed Phase D was added to the combined Phases A+B at the post-emulsification step upon cooling, i.e. at a temperature T<60° C. The stirring speed was reduced while the emulsion was cooling. Finally, the pH of the emulsion was adjusted to 5.8 by addition of an aqueous solution of sodium hydroxide (w=10%, Phase C).

Sample 4 was prepared as follows. Stearyl heptanoate, stearyl caprylate, ethylhexyl isononanoate and stearyl alcohol were heated to approx. 80° C. to form Phase A. In parallel, glyceryl stearate citrate and water were also added to approx. 80° C. to form Phase B. Carbomer was added to Phase A and homogenized, then Phase B was added to Phase A and emulsified using an Ultra Turrax stirrer. The emulsification step was carried out over a period of 2 minutes with a rotation speed of 4000 min⁻¹. The obtained emulsion was allowed to cool down using a vane stirrer. In a separate vessel, 4-isopropyl-3-methylphenol, 3-phenyl-1-propanol, decylene glycol, 4-hydroxyacetophenone and dipropylene glycol were premixed to form Phase D. The premixed Phase D was added to the combined Phases A+B at the post-emulsification step upon cooling, i.e. at a temperature T<60° C. The stirring speed was reduced while the emulsion was cooling. Finally, the pH of the emulsion was adjusted to 5.8 by addition of an aqueous solution of sodium hydroxide (w=10%, Phase C).

The resulting samples 3 and 4 were checked under the microscope the day after formulating to see if crystals are visible. For both emulsions, crystals could not be identified under the microscope.

The sample compositions 3 and 4 disclosed in Tab. 4 were tested against different microbial strains in vitro, i.e. E. coli, P. aeruginosa, S. aureus, C. albicans and A. brasiliensis. The amount of microbial species present was checked at the beginning and after 2 days, 7 days, 14 days and 28 days.

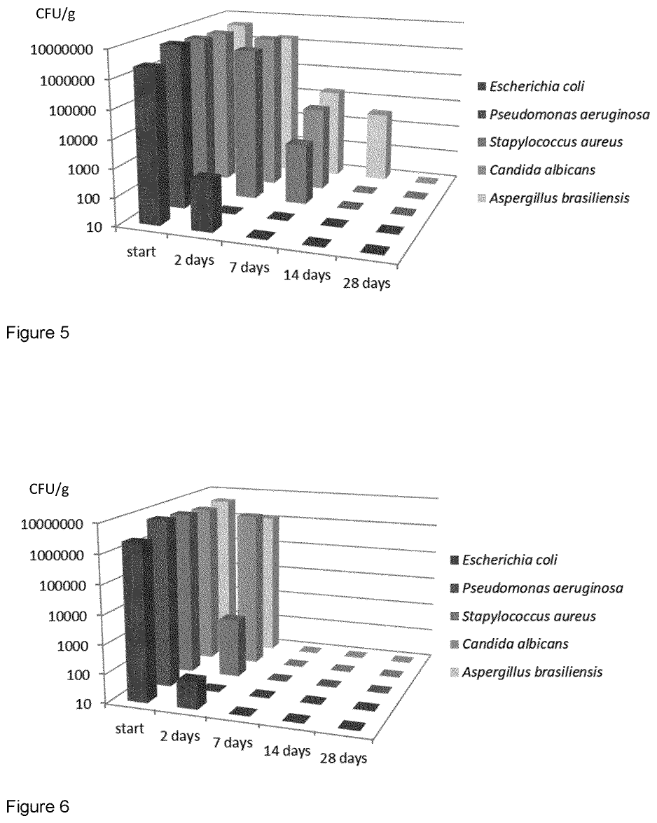

As can be seen from FIG. 5, 4-isopropyl-3-methylphenol (Sample 3) reduces the amount of the respective microbial species. The abundance of some strains, e.g. E. coli or P. aeruginosa, is already significantly reduced after 2 days. However, a full elimination of all microbial strains tested is only achieved after 28 days. In contrast, Sample 4 containing additionally 3-phenyl-1-propanol and decylene glycol shows an enhanced efficacy, since all microbial strains tested are already completely eliminated after 7 days (see FIG. 6). Thus, there is a clear synergistic effect of the components 3-phenyl-1-propanol, 4-isopropyl-3-methylphenol and decylene glycol in Sample 4.

Both formulations passed the Challenge Test, in particular, Sample 4 passed Ph. Eur.: Criteria A, whereas Sample 3 passed Ph. Eur.: Criteria B.

The aforementioned results are surprising, since the synergistic effect of Sample 4 leads to a better efficacy against the tested microbial strains than a mere increased amount of active ingredient by the factor 2.5 as in Sample 3.

### Comparative Example 2: Synergistic Effect Between 4-isopropyl-3-methylphenol, 3-phenyl-1-propanol and Decylene Glycol, a Flavouring Composition and an Antioxidant

In a further study, the synergistic effect between the flavouring composition Optamint® and the oral care composition according to the invention was examined. The synergistic effect of the antioxidant 4-hydroxyacetophenone was investigated in the same study. For this purpose, the sample compositions 5 to 7 were prepared according to Tab. 5.

Tab. 5: Sample composition comprising the flavouring mixture Optamint® as single active ingredient (Sample 5) and compositions comprising additionally 4-isopropyl-3-methylphenol, 3-phenyl-1-propanol and decylene glycol (Sample 6) or 4-isopropyl-3-methylphenol, 3-phenyl-1-propanol, decylene glycol and 4-hydroxyacetophenone (Sample 7).

| | wt.-% Material | | |
|---|---|---|---|
| | Sample | Sample | Sample |
| INCI | 5 | 6 | 7 |
| Aqua | 70.0 | 69.7 | 69.2 |
| Propylene glycol | 10.0 | 10.0 | 10.0 |
| Sorbitol | 10.0 | 10.0 | 10.0 |
| Glycerin | 5.0 | 5.0 | 5.0 |
| PEG-40 hydrogenated castor oil | 3.0 | 3.0 | 3.0 |
| Colour 1 (aq. solution of 0.01% FD&C | 1.2 | 1.2 | 1.2 |
| Blue 1 (100301)-C.I.42090 | | | |
| Flavour (Optamint ® for mouthcare) | 0.5 | 0.5 | 0.5 |
| Phenoxyethanol, | — | — | 0.5 |
| hydroxyacetophenone, | | | |
| caprylyl glycol, aqua | | | |
| Phenylpropanol, | — | 0.3 | 0.3 |
| o-cymen-5-ol, decylene glycol | | | |
| Colour 2 (aq. solution of 0.1% FD&C | 0.2 | 0.2 | 0.2 |
| Yellow 5 (656802)-C.I.19140 | | | |
| Sodium saccharin | 0.1 | 0.1 | 0.1 |

The sample compositions 5 to 7 disclosed in Tab. 5 were tested against different microbial strains in vitro, i.e. E. coli, P. aeruginosa, S. aureus, C. albicans and A. brasiliensis. The amount of microbial species present was checked at the beginning and after 2 days, 7 days, 14 days and 28 days.

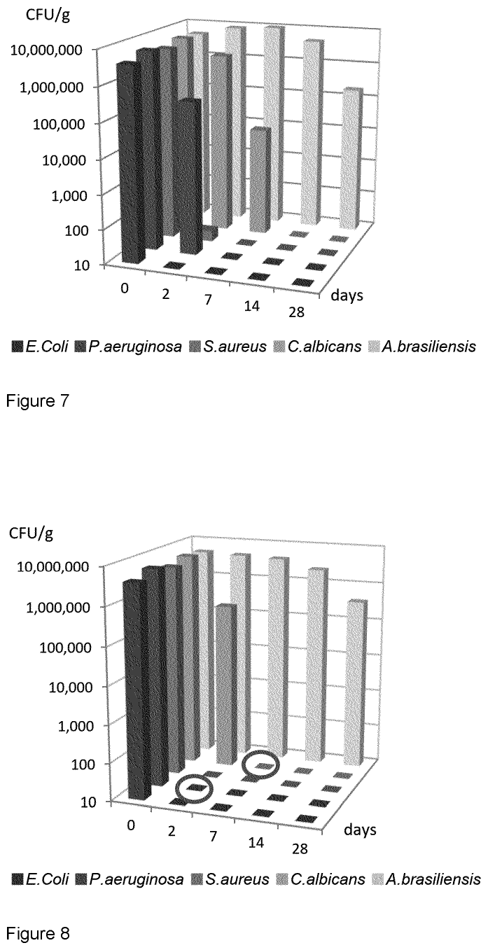

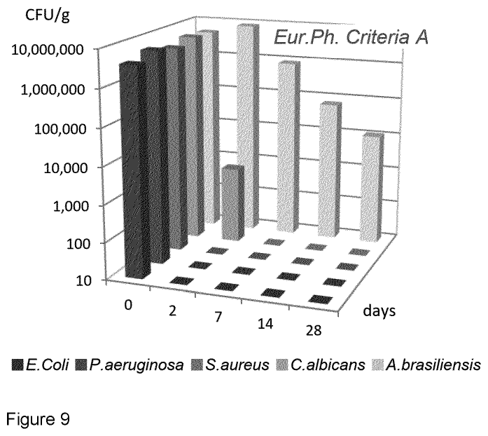

As can be seen from FIG. 7, the flavouring mixture Optamint® (Sample 5) reduces the amount of the respective microbial species. The abundance of some strains, e.g. E. coli or S. aureus, is already significantly reduced after 2 days. However, a full elimination of the microbial strains tested, except A. brasiliensis, is only achieved after 14 days. In contrast, Sample 6 containing additionally 4-isopropyl-3-methylphenol, 3-phenyl-1-propanol and decylene glycol shows an enhanced efficacy, since all microbial strains tested, except A. brasiliensis are already completely eliminated after 7 days (FIG. 8). In addition, Sample 6 shows a high efficacy against P. aeruginosa, which is one of the bacterial strains particularly related to oral malodour. A further enhanced microbial activity is achieved, if 4-hydroxyacetophenone is added (Sample 7), since the composition is then also more active against C. albicans (see FIG. 9). Thus, there are clear synergistic effects of the flavouring mixture Optamint® with the components 3-phenyl-1-propanol, 4-isopropyl-3-methylphenol and decylene glycol in Sample 6 and also with additional 4-hydroxyacetophenone in Sample 7.

Sample 7 passed the Challenge Test, in particular, Ph. Eur.: Criteria A.

The aforementioned results are surprising, since the synergistic effect of the flavouring mixture with 4-isopropyl-3-methylphenol, 3-phenyl-1-propanol and decylene glycol generally leads to a better efficacy against the tested microbial strains which is further enhanced by another synergistic effect, when 4-hydroxyacetophenone is added. In particular, 4-hydroxyacetophenone enhances the efficacy against fungi.

1. An oral care composition comprising 3-phenyl-1-propanol, an alkanediol, preferably decylene glycol, and 4-isopropyl-3-methylphenol.

2. An oral care composition comprising 4-isopropyl-3-methylphenol.

3. The oral care composition according to claim 1, for the treatment or prevention or reduction of oral malodour.

4. The oral care composition according to claim 1, wherein 4-isopropyl-3-methylphenol is present in an amount of 0.01 to 0.2 wt.-%, more preferably 0.03 to 0.08 wt.-% of the total composition.

5. The oral care composition according to claim 1, wherein 3-phenyl-1-propanol, the alkanediol, preferably decylene glycol and 4-isopropyl-3-methylphenol in sum are present in an amount of 0.01 to 0.5 wt.-% of the composition.

6. The oral care composition according to claim 1, further comprising p-hydroxyacetophenone, preferably in an amount of from 0.05 to 1.0 wt.-%.

7. The oral care composition according to claim 1, further comprising an additional alkanediol or alkanediol mixture, preferably 1,2-hexanediol and/or 1,2-octanediol, preferably in an amount of from 0.01 to 5.0 wt.-% of the total composition.

8. The oral care composition according to claim 1, further comprising hydrophilic additives, preferably such hydrophilic additives comprising propylene glycol and/or glycerol, preferably in an amount of from 0.5 to 20.0 wt.-%.

9. The oral care composition according to claim 1, further comprising a flavouring compound or fragrance, preferably in an amount of 0.1 to 1.0 wt.-%.

10. The oral care composition according to claim 1, further comprising a sweetener, preferably the sweetener comprising the sodium salt of saccharine, preferably in an amount of 0.01 to 0.5 wt.-%.

11. (canceled)

12. The oral care composition according to claim 1, for use in the treatment or prevention of oral bacterial and fungal infections, in particular preferably tooth infections and inflammations, tooth decay, dental caries, periodontal infections and inflammations, halitosis, gingivitis, candidiasis and infections with gram-negative bacterial strains, more preferably F. nucleatum and P. gingivalis.

13. The oral care composition according to claim 1, wherein the abundance of the bacterial strains F. nucleatum and P. gingivalis in the mouth is reduced by at least 40% after 4 weeks, as examined by the collection of oral saliva.

14. The oral care composition according to claim 1, wherein the oral abundance of organosulphur compounds, preferably comprising methyl mercaptan and dimethyl sulphide, is reduced by at least 40% after 4 weeks, as detected by a sulphide detector.

15. The oral care composition according to claim 1, wherein the oral care composition is a component of a mouthwash, cosmetic mouthwash, toothpaste, tooth gel, subgingival gel component or mouth odour reducing agent.

16. The oral care composition according to claim 2, for the treatment or prevention or reduction of oral malodour.

17. The oral care composition according to claim 2, wherein 4-isopropyl-3-methylphenol is present in an amount of 0.01 to 0.2 wt.-%, more preferably 0.03 to 0.08 wt.-% of the total composition.

18. The oral care composition according to claim 2, further comprising one or more of:
p-hydroxyacetophenone, preferably in an amount of from 0.05 to 1.0 wt.-%;
an alkanediol or alkanediol mixture, preferably 1,2-hexanediol and/or 1,2-octanediol, preferably in an amount of from 0.01 to 5.0 wt.-% of the total composition;
hydrophilic additives, preferably such hydrophilic additives comprising propylene glycol and/or glycerol, preferably in an amount of from 0.5 to 20.0 wt.-%;
a flavouring compound or fragrance, preferably in an amount of 0.1 to 1.0 wt.-%; and
a sweetener, preferably the sweetener comprising the sodium salt of saccharine, preferably in an amount of 0.01 to 0.5 wt.-%.

19. The oral care composition according to claim 2, for the treatment or prevention of oral bacterial and fungal infections, in particular preferably tooth infections and inflammations, tooth decay, dental caries, periodontal infections and inflammations, halitosis, gingivitis, candidiasis and infections with gram-negative bacterial strains, more preferably F. nucleatum and P. gingivalis.

20. The oral care composition according to claim 2, wherein at least one of:
the abundance of the bacterial strains F. nucleatum and P. gingivalis in the mouth is reduced by at least 40% after 4 weeks, as examined by the collection of oral saliva; and
the oral abundance of organosulphur compounds, preferably comprising methyl mercaptan and dimethyl sulphide, is reduced by at least 40% after 4 weeks, as detected by a sulphide detector.

21. The oral care composition according to claim 2, wherein the oral care composition is a component of a mouthwash, cosmetic mouthwash, toothpaste, tooth gel, subgingival gel or mouth odour reducing agent.
